(19) United States
(12) Patent Application Publication
(10) Pub. No.: US 2025/0269357 A1
(43) Pub. Date: Aug. 28, 2025

(54) SELECTIVE HYDROGENATION COPPER-BASED CATALYST WITH EXCELLENT THERMAL STABILITY AND ITS PREPARATION METHOD

(71) Applicants: Beijing University of Chemical Technology, Beijing (CN); Quzhou Resource and Chemical Innovation Research Institute, Quzhou City (CN)

(72) Inventors: JunTing Feng, Beijing (CN); Qian Wang, Beijing (CN); FengYu Zhang, Beijing (CN); DianQing Li, Beijing (CN); XiaoXiao Guo, Beijing (CN); Xue Duan, Beijing (CN)

(21) Appl. No.: 18/641,668

(22) Filed: Apr. 22, 2024

(30) Foreign Application Priority Data
Feb. 22, 2024 (CN) 202410199568.4

(51) Int. Cl. B01J 23/86; B01J 23/02; B01J 23/825; B01J 37/00; B01J 37/02; B01J 37/03; B01J 37/04; B01J 37/06; B01J 37/08; B01J 37/14; B01J 37/18

(52) U.S. Cl. CPC B01J 23/868; B01J 23/02; B01J 23/825; B01J 37/009; B01J 37/0236; B01J 37/038; B01J 37/04; B01J 37/06; B01J 37/08; B01J 37/14; B01J 37/18

(57) ABSTRACT

Cu-based catalysts with excellent thermal stability for selective hydrogenation and a preparation method thereof are provided. The method includes: first, six mixed metal salts are instantaneously nucleated in an alkaline solution by the nucleation/crystallization isolation method, to prepare high-entropy composite metal hydroxides (H-LDHs) with uniform element distribution and similar metal proportions. Furthermore, well-crystallized high-entropy oxides (HEOs) are obtained based on the structural topology characteristics of H-LDHs, and a series of Cu-Mx/HEOs catalysts with flexible adjustable catalytic microzone geometries and electronic structures are obtained using the HEOs as precursors.

### CROSS-REFERENCE TO RELATED APPLICATIONS

This application which claims priority to Chinese Patent Application No. 2024101995684, filed on Feb. 22, 2024, The aforementioned applications are incorporated herein by reference in their entireties.

### TECHNICAL FIELD

The application relates to a catalyst for selective hydrogenation of carbon-carbon triple bonds and its preparation method. It belongs to the fields of petrochemical and fine chemical technology.

### BACKGROUND

Ethylene is an important substance in various industrial productions, especially in the polymer industry, where its production signifies the level of a country's petrochemical industry. According to statistics, over 200 million tons of ethylene and propylene are produced in the world every year. Ethylene is produced by steam-mediated high hydrocarbon cracking in high-temperature furnaces. However, the products produced by this process contain about 0.1-1% acetylene, which easily causes poisoning and deactivation of catalysts used in downstream ethylene polymerization reactions and thus affects the yield of polyethylene.

In recent years, global precious metal reserves are decreasing, and the supply gap is increasing year by year, and the price is rising rapidly. Thus it is significant to find effective substitutes for precious metals in petrochemical processes. Copper with only one electron in its 4 s orbital exhibits certain hydrogenation activity and excellent selectivity for olefin. Shi has explored the effects of copper's geometric structure on acetylene hydrogenation performance using atomic layer deposition to prepare copper single atoms and a series of copper nanoparticles (ACS Catal. 2020, 10(5): 3495-3504). Fu has also investigated the modification of Cu interface structures with FeyMgOx with different Cu/Fe ratios (ACS Catal. 2021, 11, 17, 11117-11128). However, copper nanoparticles have a low Tamman temperature and are prone to aggregation and growth, leading to irreversible catalyst deactivation and significantly affecting catalyst stability. Particularly in exothermic reactions and strongly exothermic reactions at high reaction temperatures, strict reaction conditions pose great challenges to the stability of Cu-based catalysts. The phenomenon of strong metal-support interaction (SMSI) occurs when the surface of supported metal catalysts undergoes a classic metal-carrier interaction, the carrier species migrate to the surface of the metal nanoparticles to wrap them and inhibit their aggregation and growth. Thereby this method provides a unique solution to improve the catalyst's lifetime and has been widely applied. However, the physicochemical properties of copper itself make it difficult for Cu-based catalysts to form SMSI since copper has a low work function to make it difficult to accept electrons, and weak ability to activate and dissociate hydrogen gas hinders carrier reduction to make it difficult to meet the necessary conditions of SMSI formation. Therefore, it is crucial and yet challenging to design catalyst structures to enhance Cu-based catalyst stability.

In recent years, high-entropy oxides have received widespread attention due to their unique structure and versatile chemical properties. Based on their compositional adjustability, various elements can be simultaneously incorporated into high-entropy oxides to obtain catalyst precursor structures with variable compositions. Lattice distortion effects and the electronic structure diversity of high-entropy components can enhance the activity of catalytic material. Entropy-driven structural stability can improve catalyst long-term performance, and the unique hysteresis effect of high-entropy materials can slow down or prevent the aggregation of catalytic active metals, thereby catalyst structural stability and material integrity are enhanced. Layered double hydroxides (LDHs) are two-dimensional layered materials similar to hydrotalcite, where metals are uniformly dispersed within the layers and can easily convert to oxides at low temperatures, and present unique advantages in preparing high-entropy oxide materials. Therefore, the present invention aims to activate partial metals in high-entropy oxides used as precursors and to construct catalysts with heat dissipation functionality due to their multi-component and entropy-stabilized structural characteristics, enabling catalytic active metals to have excellent long-term performance under harsh conditions.

### SUMMARY

The purpose of the present invention is to provide a Cu-based catalyst with excellent thermal stability for selective hydrogenation and its preparation method. The catalyst is primarily used for the selective hydrogenation of carbon-carbon triple bonds.

The catalyst provided by the present invention is represented as Cu-Mx/HEOs, where Cu is the active component, M is a reducible auxiliary metal selected from Zn, Fe, Co, Cr, Ni, Ga, Ti, Mn, with Co or Fe being preferred, and x represents the proportion of M relative to the active metal Cu, where x=1-5. HEOs represents the Cu-based high-entropy oxide carrier, wherein the elemental composition in the carrier is Cu and M1-5 which denotes a mixture of any five of Zn, Mg, Co, Ga, Al, Fe, Mn, Ni, and Cr, with Zn, Co, Fe, Ga, and Al being preferred.

The preparation method of the Cu-Mx/HEOs catalyst for selective hydrogenation is as follows:
(1) Dissolve M²⁺ and M³⁺ metal salts with an M²⁺/M³⁺ ion molar ratio of 2-3:1 in deionized water to prepare a mixed salt solution, wherein the M²⁺ salts is a mixture of three types of salts, in which Cu salts is indispensable, further the M²⁺ salts is a mixture of Cu(NO₃)₂·3H₂O and any two of Zn(NO₃)₂·6H₂O, Mg(NO₃)₂·6H₂O, Co(NO₃)₂·6H₂O, Ni(NO₃)₂·6H₂O; the M³⁺ salts is a mixture of any three types of salts seleced from Ga(NO₃)₃·3H₂O, Al(NO₃)₃·9H₂O, Fe(NO₃)₃·9H₂O, Mn(NO₃)₄·4H₂O, and Cr(NO₃)₃·9H₂O; the total concentration of metal ions in the mixed salt solution is 0.12-0.36 mol·1⁻¹, wherein Cu ions account for 15-20% of the total molar amount of metal ions, the other two types of M²⁺ metal ions have similar concentrations, and together account for 35-40% of the total molar amount of metal ions, and the remaining three types of M³⁺ metal ions have similar concentrations, and together account for 40-50% of the total molar amount of metal ions;
furthermore dissolve a mix of any two of NaOH, KOH, Na2CO3, or NaHCO3 in deionized water to prepare an alkaline solution with a concentration of 0.12-0.36 mol·1⁻¹.
(2) Start nucleation reactor referring to patent CN201210105567.6, and set the stator-rotor gap of the nucleation reactor to 0.1-1 mm and the speed to 1000-3000 rpm, feed the mixed salt solution and the alkaline solution in step (1) into the reactor at the same rate of 10-30 mL·min⁻¹ with a peristaltic pump to nucleate rapidly and control the total number of metal cations in the salt solution to be equal to the number of anions in the alkaline solution, and collect the nucleation slurry at the outlet.
(3) The nucleation slurry is transferred to a crystallization vessel, to be crystallized and grow at 60-120° C. for 6-18 hours. Then cool naturally to room temperature, and the crystallization product is centrifuged and washed with deionized water to neutrality, dried in a freeze dryer for 12-24 hours to obtain hexa-element layered composite metal hydroxide high-entropy hydrotalcite denoted as HEHs. Use ICP to determine the element content of the obtained HEHs and calculate its entropy value. The entropy value is calculated using the formula Sconfig=−R[(Σᵢ₌₁ⁿxᵢlnxᵢ)_{cation}. When Sconfig>1.5 R, it can be considered that high-entropy hydrotalcite has been successfully synthesized.
(4) The HEHs obtained in step (3) is heated with a heating rate of 5-10° C.·min⁻¹ to 400-800° C. in air atmosphere and calcined for 3-6 hours to obtain hexa-element high-entropy oxides denoted as HEOs.
(5) The HEHs obtained in step (4) is heated with a rate of 2-10° C./min to 400-800° C. and reduced in a 10-20 vol. H2/N2 atmosphere for 3-6 hours to obtain Cu-Mx/HEOs catalysts.

Due to the different reduction potentials or different metals and their reduction degrees being proportional to the reduction temperature and time, the required interface structure Cu-Mx catalyst can be prepared by controlling the reduction temperature and time, selecting the types and proportions of M in the carrier, wherein the Cu-Co interface structure catalyst has better application effects.

Preferably, in step (1), the molar ratio of M²⁺/M³⁺ metal ions in the mixed salt solution is 2-2.2:1.

The characteristic of this preparation method is the use of a nucleation/crystallization isolation method to rapidly nucleate a mixed solution of six metal salts and an alkaline solution, and to strengthen the crystal nucleation environment by adjusting key parameters such as the stator-rotor gap and rotation speed of the nucleation reactor, to synthesize composite metal hydroxides (LDHs) with controllable primary particle size. The fast shear in the nucleation/crystallization isolation method allows rapid mixing of the six metal salt solutions, and breaks through the limitation of different metal Ksp, and enables the uniform dispersion of metal elements in the hydrotalcite layer. By performing structure detection and entropy value calculation on the synthesized LDHs, the configurational entropy of the LDHs exceeds 1.5 R, which meets the standard of high-entropy materials. Furthermore, Cu-based high-entropy oxides (HEOs) with controllable spinel crystal structure, size, and specific surface area were successfully prepared utilizing the topological effect of LDHs at lower temperatures. Then, the Cu-Mx/HEOs catalyst is prepared by controlling the reduction conditions using the above-mentioned HEOs as precursors. The proportion of the main metal Cu and the guest metal M in the interface structure of the catalyst is adjustable. Furthermore, the material exhibits the entropy-stabilizing characteristic unique to high-entropy materials. The Cu-based catalyst prepared by this method exhibits excellent catalytic activity and long-term performance in strong exothermic hydrogenation reactions.

### BRIEF DESCRIPTION OF THE DRAWINGS

FIG. 1 is the X-ray diffraction (XRD) spectrum of the catalyst precursor HEOs prepared in embodiment 1, indicating a well-defined spinel crystal structure.

FIG. 2 is the HR-TEM image of the catalyst prepared in embodiment 1, indicating a well-defined carrier crystal structure with some metals reduced to particles.

FIG. 3 is the Cu 2p XPS spectrum of the catalyst prepared in embodiment 1, indicating that most of the Cu in the carrier is reduced to Cu0/Cu+.

FIG. 4 is the experimental results of the acetylene selective hydrogenation reaction using the catalyst prepared in embodiment 1, wherein A is the curve of acetylene conversion rate versus temperature, and B is the curve of acetylene selectivity versus acetylene conversion rate.

FIG. 5 is the stability results of the acetylene selective hydrogenation reaction using the catalyst prepared in embodiment 1, wherein A is the curve of acetylene conversion rate versus time, and B is the curve of acetylene selectivity versus time.

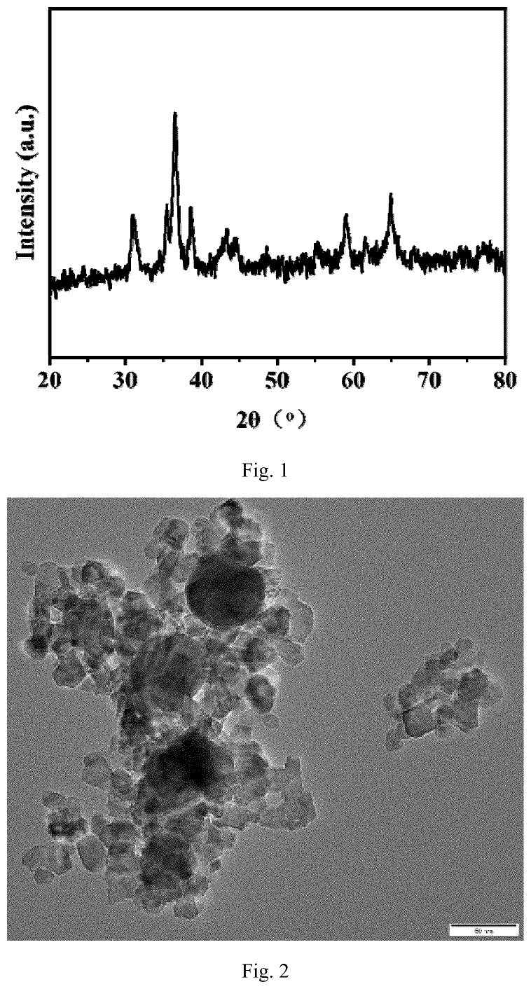

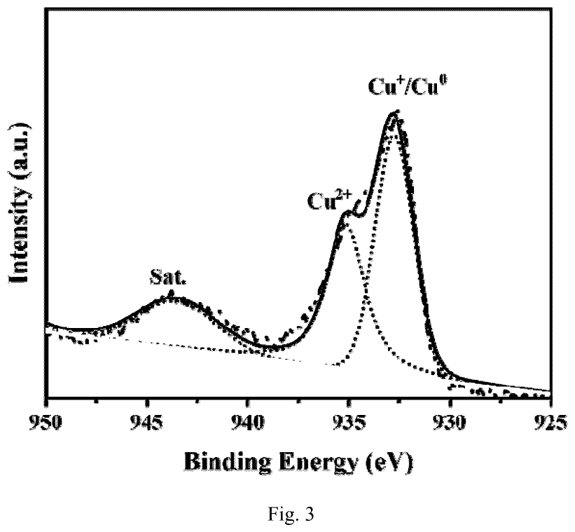

### DETAILED DESCRIPTION OF THE EMBODIMENTS

In order to better illustrate the technological process and technical advantages of the present application, the following embodiments are used to illustrate the effect of the application. But the present application is not limited to the content of the embodiment.

Unless otherwise specified, percentages in embodiments of the present application are percentages by mass; The raw materials used are all conventional types of raw materials in the field that can be purchased in the market; The methods used are all conventional methods in the art (including detection methods); The equipment used is all conventional equipment in the field.

### Embodiment 1

(1) Dissolve 1.64 g Cu(NO₃)₂·3H₂O, 2.36 g Zn(NO₃)₂·6H₂O, 2.05 g Mg(NO₃)₂·6H₂O, 1.62 g Fe(NO₃)₃·9H₂O, 1.02 g Ga(NO₃)₃·3H₂O, and 1.5 g Al(NO₃)₃·9H₂O in 100 ml deionized water to prepare a mixed salt solution; dissolve 2.03 g Na₂CO₃ and 1.84 g NaOH in 100 ml deionized water to prepare an alkali solution.
(2) Start the nucleation reactor, set the stator-rotor gap to 0.2 mm, and the speed to 3000 rpm. Use a peristaltic pump to deliver the mixed salt solution and alkali solution prepared in step (2) to the reactor at a rate of 20 ml/min for rapid nucleation, and collect the nucleation slurry at the slurry outlet.
(3) Add the nucleation slurry to a three-neck flask, crystallize and grow at 60° C. in water bath for 12 hours, naturally cool to room temperature, centrifuge to separate the crystallization product, and wash with deionized water to neutrality. Freeze-dry the product for 12 hours to obtain the layered composite metal high-entropy hydroxide precursor CuZnMgFeGaAl-LDHs, with an entropy value of 1.65 R.
(4) The CuZnMgFeGaAl-LDHs precursor obtained in step (3) is heated to 600° C. at a rate of 5° C./min in air atmosphere and calcined for 4 hours to obtain the corresponding composite metal high-entropy oxide CuZnMgFeGaAl-HEOs.
(5) Reduce the CuZnMgFeGaAl-HEOs obtained in step (4) to 600° C. at a rate of 10° C./min in a 10 vol. % H₂/N₂ atmosphere for 4 hours. Some Cu and Fe in the precursor are partially reduced, and the catalyst Cu-Fe/HEOs with a Cu/Fe ratio of 1:1 is obtained.

Apply the prepared catalyst to acetylene selective hydrogenation reaction experiments as the following steps:

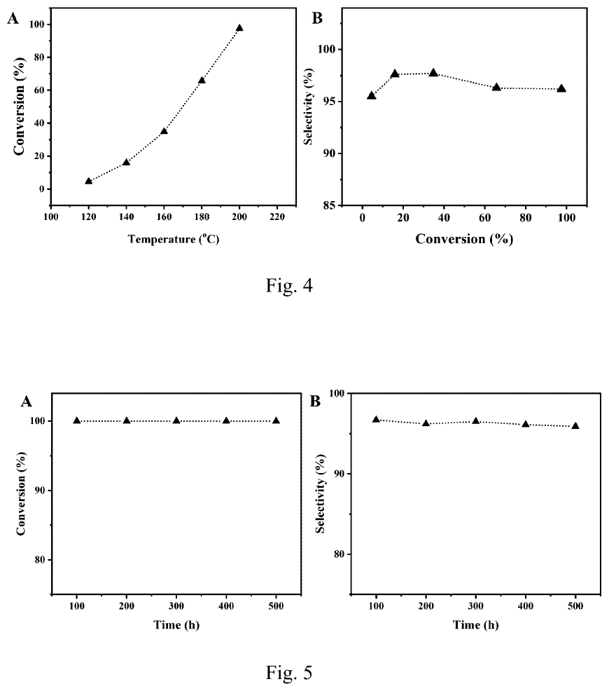

Mix 200 mg catalyst with 1.8 g quartz sand with a particle size of 40-70 mesh and load it into a quartz reaction tube with a diameter of 10 mm. Before the reaction, activate the sample at 150° C. in a 10 vol. % H₂/N₂ mixed gas for 2 hours and naturally cool to room temperature. The testing temperature ranges from 100 to 220° C., with the gas composition of 0.71% acetylene and 2.86% hydrogen and 70.72% ethylene and balanced nitrogen. The test pressure is 1 bar, and the space velocity is 3800 h−1. The composition and content of reactants and products are analyzed by gas chromatography, and data processing is performed by normalization. To ensure test accuracy, results are recorded after maintaining the specified temperature for 30 minutes. Three sets of tests are conducted, and the average value represents the catalytic performance data at that temperature, as shown in FIG. 4. When the catalyst performance is stable during the tests, a long-term stability evaluation is performed. Tests are conducted every 100 hours when the acetylene conversion rate is 100% and the ethylene selectivity is >95%, for a total of 500 hours, as shown in FIG. 5.

### Embodiment 2

(1) Dissolve 1.64 g Cu(NO₃)₂·3H₂O, 2.36 g Zn(NO₃)₂·6H₂O, 2.05 g Mg(NO₃)₂·6H₂O, 1.62 g Fe(NO₃)₃·9H₂O, 1.02 g Ga(NO₃)₃·3H₂O, and 1.5 g Al(NO₃)₃·9H₂O in 100 ml deionized water to prepare a mixed salt solution; dissolve 2.03 g Na₂CO₃ and 2.36 g KOH in 100 ml deionized water to prepare an alkali solution.
(2) Start the nucleation reactor, set the stator-rotor gap to 0.1 mm, and the speed to 1500 rpm. Use a peristaltic pump to respectively deliver the mixed salt solution and alkali solution from step (1) into the reactor at a rate of 30 ml/min for rapid nucleation, and collect the nucleation slurry at the slurry outlet.
(3) Add the nucleation slurry to a three-neck flask, crystallize and grow at 80° C. in water bath for 18 hours, naturally cool to room temperature, centrifuge to separate the crystallization product, and wash with deionized water to neutrality. Freeze-dry the product for 24 hours to obtain the layered composite metal high-entropy hydroxide precursor CuZnMgFeGaAl-LDHs, with an entropy value of 1.55 R.
(4) The CuZnMgFeGaAl-LDHs precursor obtained in step (3) is heated to 600° C. at a rate of 5° C./min in air atmosphere and calcined for 3 hours to obtain the corresponding composite metal high-entropy oxide CuZnMgFeGaAl-HEOs.
(5) Reduce the CuZnMgFeGaAl-HEOs obtained in step (4) to 800° C. at a rate of 10° C./min in a 10 vol. % H₂/N₂ atmosphere for 5 hours. Cu and Fe in the precursor are partially reduced, and the catalyst Cu-Fe/HEOs with a Cu/Fe ratio of 1:2 is obtained.

### Embodiment 3

(1) Dissolve 1.64 g Cu(NO₃)₂·3H₂O, 2.38 g Co(NO₃)₂·6H₂O, 2.05 g Mg(NO₃)₂·6H₂O, 1.62 g Fe(NO₃)₃·9H₂O, 1.02 g Ga(NO₃)₃·3H₂O, and 1.5 g Al(NO₃)₃·9H₂O in 100 ml deionized water to prepare a mixed salt solution; dissolve 2.03 g Na₂CO₃ and 1.84 g NaOH in 100 ml deionized water to prepare an alkali solution.
(2) Start the nucleation reactor, set the stator-rotor gap to 0.2 mm, and the speed to 3000 rpm. Use a peristaltic pump to respectively deliver the mixed salt solution and alkali solution from step (1) into the reactor at a rate of 20 ml/min for rapid nucleation, and collect the nucleation slurry at the slurry outlet.
(3) Add the nucleation slurry to a three-neck flask, crystallize and grow at 70° C. in water bath for 6 hours, naturally cool to room temperature, centrifuge to separate the crystallization product, and wash with deionized water to neutrality. Freeze-dry the product for 12 hours to obtain the layered composite metal high-entropy hydroxide precursor CuCoMgFeGaAl-LDHs, with an entropy value of 1.70 R.
(4) The CuCoMgFeGaAl-LDHs precursor obtained in step (3) is heated to 600° C. at a rate of 5° C./min in air atmosphere and calcined for 3 hours to obtain the corresponding composite metal high-entropy oxide CuCoMgFeGaAl-HEOs.
(5) Reduce the CuCoMgFeGaAl-HEOs obtained in step (4) to 600° C. at a rate of 10° C./min in a 10 vol. % H₂/N₂ atmosphere for 6 hours. Cu and Co in the precursor are partially reduced, and the catalyst Cu-Co/HEOs with a Cu/Co ratio of 1:1 is obtained.

### Embodiment 4

(1) Dissolve 1.64 g Cu(NO₃)₂·3H₂O, 2.35 g/Ni(NO₃)₂·6H₂O, 2.05 g Mg(NO₃)₂·6H₂O, 1.62 g Fe(NO₃)₃·9H₂O, 1.02 g Ga(NO₃)₃·3H₂O, and 1.5 g Al(NO₃)₃·9H₂O in 100 ml deionized water to prepare a mixed salt solution; dissolve 2.03 g Na₂CO₃ and 1.84 g NaOH in 100 ml deionized water to prepare an alkali solution.
(2) Start the nucleation reactor, set the stator-rotor gap to 0.2 mm, and the speed to 3000 rpm. Use a peristaltic pump to respectively deliver the mixed salt solution and alkali solution from step (1) into the reactor at a rate of 20 ml/min for rapid nucleation, and collect the nucleation slurry at the slurry outlet.
(3) Add the nucleation slurry to a three-neck flask, crystallize and grow at 70° C. for 6 hours, naturally cool to room temperature, centrifuge to separate the crystallization product, and wash with deionized water to neutrality. Freeze-dry the product for 12 hours to obtain the layered composite metal high-entropy hydroxide precursor CuMgNiFeGaAl-LDHs, with an entropy value of 1.65 R.
(4) The CuMgNiFeGaAl-LDHs precursor obtained in step (3) is heated to 600° C. at a rate of 5° C./min in air atmosphere and calcined for 4 hours to obtain the corresponding composite metal high-entropy oxide CuMgNiFeGaAl-HEOs.
(5) Reduce the CuMgNiFeGaAl-HEOs obtained in step (4) to 600° C. at a rate of 10° C./min in a 10 vol. % H₂/N₂ atmosphere for 5 hours. Cu and Ni in the precursor are partially reduced, and the catalyst Cu-Ni/HEOs with a Cu/Fe ratio of 1:1 is obtained.

### Embodiment 5

(1) Dissolve 1.64 g Cu(NO₃)₂·3H₂O, 2.35 g Ni(NO₃)₂·6H₂O, 2.05 g Mg(NO₃)₂·6H₂O, 1.62 g Fe(NO₃)₃·9H₂O, 1.62 g Cr(NO₃)₃·3H₂O, and 1.5 g Al(NO₃)₃·9H₂O in 100 ml deionized water to prepare a mixed salt solution; dissolve 2.03 g Na₂CO₃ and 1.84 g NaOH in 100 ml deionized water to prepare an alkali solution.
(2) Start the nucleation reactor, set the stator-rotor gap to 0.2 mm, and the speed to 3000 rpm. Use a peristaltic pump to respectively deliver the mixed salt solution and alkali solution from step (1) into the reactor at a rate of 20 ml/min for rapid nucleation, and collect the nucleation slurry at the slurry outlet.
(3) Add the nucleation slurry to a three-neck flask, crystallize and grow at 70° C. for 6 hours, naturally cool to room temperature, centrifuge to separate the crystallization product, and wash with deionized water to neutrality. Freeze-dry the product for 12 hours to obtain the layered composite metal high-entropy hydroxide precursor CuMgNiFeCrAl-LDHs, with an entropy value of 1.75 R.
(4) The CuMgNiFeCrAl-LDHs precursor obtained in step (3) is heated to 600° C. at a rate of 5° C./min in air atmosphere and calcined for 5 hours to obtain the corresponding composite metal high-entropy oxide CuMgNiFeCrAl-HEOs.
(5) Reduce the CuMgNiFeCrAl-HEOs obtained in step (4) to 800° C. at a rate of 10° C./min in a 10 vol. % H₂/N₂ atmosphere for 5 hours. Cu and Ni in the precursor are partially reduced, and the catalyst Cu-Ni/HEOs with a Cu/Fe ratio of 1:2 is obtained.

What is claimed is:

1. A method for preparing Cu-based selective hydrogenation catalysts, comprising steps as follows:
(1) Dissolving M²⁺ and M³⁺ metal salts with an M²⁺/M³⁺ ion molar ratio of 2-3:1 in deionized water to prepare a mixed salt solution, wherein the M²⁺ salts is a mixture of three types of salts, in which Cu salts is indispensable, further the M²⁺ salts is a mixture of Cu(NO₃)₂·3H₂O and any two of Zn(NO₃)₂·6H₂O, Mg(NO₃)₂·6H₂O, Co(NO₃)₂·6H₂O, Ni(NO₃)₂·6H₂O; the M³⁺ salts is a mixture of any three types of salts seleced from Ga(NO₃)₃·3H₂O, Al(NO₃)₃·9H₂O, Fe(NO₃)₃·9H₂O, Mn(NO₃)₄·4H₂O, and Cr(NO₃)₃·9H₂O; the total concentration of metal ions in the mixed salt solution is 0.12-0.36 mol·1⁻¹, wherein Cu ions account for 15-20% of the total molar amount of metal ions, the other two types of M²⁺ metal ions have similar concentrations, and together account for 35-40% of the total molar amount of metal ions, and the remaining three types of M³⁺ metal ions have similar concentrations, and together account for 40-50% of the total molar amount of metal ions;
furthermore dissolving a mix of any two of NaOH, KOH, Na₂CO₃, or NaHCO₃ in deionized water to prepare an alkaline solution with a concentration of 0.12-0.36 mol·1⁻¹;
(2) starting nucleation reactor, setting the stator-rotor gap of the nucleation reactor to 0.1-1 mm and the speed to 1000-3000 rpm, feeding the mixed salt solution and the alkaline solution in step (1) into the reactor at the same rate of 10-30 ml·min⁻¹ with a peristaltic pump to nucleate rapidly and controling the total number of metal cations in the salt solution to be equal to the number of anions in the alkaline solution, and collecting the nucleation slurry at the outlet;
(3) transfering the nucleation slurry to a crystallization vessel, and crystallizing and growing at 60-120° C. for 6-18 hours, cooling naturally to room temperature, centrifuging and washing the crystallization product with deionized water to neutrality, drying in a freeze dryer for 12-24 hours to obtain hexa-element layered composite metal hydroxide high-entropy hydrotalcite denoted as HEHs, with a configurational entropy value Sconfig>1.5 R;
(4) heating the HEHs obtained in step (3) with a heating rate of 5-10° C.·min⁻¹ to 400-800° C. in air atmosphere and calcining for 3-6 hours to obtain hexa-element high-entropy oxides denoted as HEOs;
(5) heating with a rate of 2-10° C./min to 400-800° C. and reducing the HEHs obtained in step (4) in a 10-20 vol. H₂/N₂ atmosphere for 3-6 hours to obtain Cu-Mx/HEOs catalysts.

2. The preparation method according to claim 1, wherein in step (1), the molar ratio of M²⁺/M³⁺ metal ions in the mixed salt solution is 2-2.2:1.

3. Selective hydrogenation Cu-based catalysts prepared by the preparation method according to claim 1, wherein the catalysts are represented as Cu-Mx/HEOs, where Cu is the active component, M is a reducible auxiliary metal selected from Zn, Fe, Co, Cr, Ni, Ga, Ti, and Mn, x represents the ratio of M to active metal Cu, x is 1 to 5, and HEOs represents the Cu-based high-entropy oxide carrier, wherein the elemental composition in the carrier is Cu and M₁₋₅ which denotes a mixture of any five of Zn, Mg, Co, Ga, Al, Fe, Mn, Ni, and Cr.

4. Selective hydrogenation Cu-based catalysts according to claim 3, wherein in Cu-Mx/HEOs, M is Co or Fe; M₁₋₅ is a mixture of Zn, Co, Fe, Ga, and Al.
